(19) United States
(12) Patent Application Publication
(10) Pub. No.: US 2023/0096407 A1
(43) Pub. Date: Mar. 30, 2023

(54) ULTRASONIC TRANSDUCER, ULTRASONIC PROBE, ULTRASONIC DIAGNOSTIC APPARATUS, AND METHOD FOR MANUFACTURING ULTRASONIC TRANSDUCER

(71) Applicant: Konica Minolta Inc., Tokyo (JP)

(72) Inventor: KIYOKAZU MORITA, Tokyo (JP)

(21) Appl. No.: 17/891,570

(22) Filed: Aug. 19, 2022

(30) Foreign Application Priority Data
Sep. 6, 2021 (JP) 2021-144735

(51) Int. Cl. A61B 8/00; B32B 7/12; B32B 25/08; B32B 27/08; B32B 27/16; B06B 1/06; G10K 11/02

(52) U.S. Cl. CPC A61B 8/4483; B32B 7/12; B32B 25/08; B32B 27/08; B32B 27/16; B06B 1/067; G10K 11/02; B32B 25/20

(57) ABSTRACT

An ultrasonic transducer includes: a laminate in which a plurality of acoustic members is laminated; and an adhesive layer that includes a silane coupling agent and an adhesive, the adhesive layer joining any two of the plurality of acoustic members to each other, wherein the silane coupling agent has a structure represented by general formula (1):

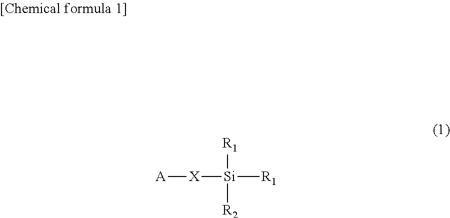

wherein R₁s each independently represent a methoxy group or an ethoxy group, R₂ represents a methoxy group, an ethoxy group, or a hydrogen atom, X represents a linear or branched organic chain having 4 or more continuous carbon atoms, and A represents a reactive functional group.

The entire disclosure of Japanese patent Application No. 2021-144735, filed on Sep. 6, 2021, is incorporated herein by reference in its entirety.

### BACKGROUND

### Technological Field

The present invention relates to an ultrasonic transducer, an ultrasonic probe, an ultrasonic diagnostic apparatus, and a method for manufacturing the ultrasonic transducer.

### Description of the Related Art

An ultrasonic probe is used for obtaining the shape, movement, or the like of a living tissue as a diagnostic image by simple operation of applying the ultrasonic probe connected to an ultrasonic diagnostic apparatus or communicable with the ultrasonic diagnostic apparatus to a body surface or inserting the ultrasonic probe into the body.

The ultrasonic probe incorporates an ultrasonic transducer or the like for transmitting and receiving an ultrasonic wave. The ultrasonic transducer has a laminate in which a plurality of acoustic members such as a piezoelectric material and an acoustic matching layer is laminated, and many of the plurality of acoustic members are bonded to each other with an adhesive or the like. In such an ultrasonic transducer, when an adhesive or the like is peeled off, transmission and reception of an ultrasonic wave is not normally performed, accuracy of a diagnostic image is deteriorated, and therefore the ultrasonic transducer is not suitable for use. Therefore, it is required to improve adhesive strength between the acoustic members such as a piezoelectric material and a matching layer.

As a method for enhancing the adhesive strength between the acoustic members, it is known to use a silane coupling agent.

For example, JP 2003-284192 A discloses an ultrasonic probe in which a gold electrode of a piezoelectric material is bonded to a resin layer (acoustic matching layer) by applying a silane coupling agent containing γ-mercaptopropyltrimethoxysilane as a main component and an adhesive to an adhesive surface therebetween. JP 2003-284192 A discloses that a defect such as peeling of the resin layer from the gold electrode can be suppressed, and stable ultrasonic characteristics can be obtained.

JP 2005-139458 A discloses an ultrasonic transducer in which a gold surface with which a piezoelectric material is coated is surface-treated with a solution containing a sulfur-containing alkoxysilane, an adhesive containing a sulfur-containing alkoxysilane is applied to the gold surface and an acoustic impedance layer, and the gold surface and the acoustic impedance layer are bonded to each other. JP 2005-139458 A describes that adhesive strength between the gold surface and the acoustic impedance layer can be enhanced by treating the gold surface with a solution containing a sulfur-containing alkoxysilane and then applying the adhesive containing a sulfur-containing alkoxysilane.

However, even when an ultrasonic transducer is manufactured using the silane coupling agent described in JP 2003-284192 A or the silane coupling agent described in JP 2005-139458 A together with an adhesive, adhesive strength between laminated acoustic members cannot be sufficiently ensured in some cases.

Therefore, when dicing is performed at the time of manufacturing an ultrasonic transducer, one acoustic member bonded may be peeled off from the other acoustic member due to stress applied to the laminate of the plurality of acoustic members.

In addition, an ultrasonic probe incorporating an ultrasonic transducer is used in contact with a human body as described above, and therefore it is necessary to disinfect and clean the ultrasonic probe, for example, by immersing the ultrasonic probe in a disinfection liquid after use. When the ultrasonic probe is disinfected and cleaned, a chemical solution may infiltrate into the ultrasonic probe to deteriorate adhesive strength. Therefore, one of bonded acoustic members may be peeled off from the other acoustic member.

### SUMMARY

The present invention has been made in view of the above circumstances, and an object of the present invention is to provide an ultrasonic transducer capable of improving adhesive strength between acoustic members, and suppressing peeling of the acoustic members at the time of dicing and peeling of the acoustic members at the time of disinfection and cleaning, an ultrasonic probe including the ultrasonic transducer, an ultrasonic diagnostic apparatus including the ultrasonic probe, and a method for manufacturing the ultrasonic transducer.

To achieve the abovementioned object, according to an aspect of the present invention, an ultrasonic transducer reflecting one aspect of the present invention comprises: a laminate in which a plurality of acoustic members is laminated; and an adhesive layer that comprises a silane coupling agent and an adhesive, the adhesive layer joining any two of the plurality of acoustic members to each other, wherein the silane coupling agent has a structure represented by general formula (1):

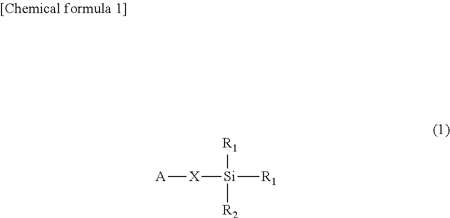

wherein R₁s each independently represent a methoxy group or an ethoxy group, R₂ represents a methoxy group, an ethoxy group, or a hydrogen atom, X represents a linear or branched organic chain having 4 or more continuous carbon atoms, and A represents a reactive functional group.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a cross-sectional view illustrating an example of an entire structure of an ultrasonic transducer according to an embodiment of the present invention;

FIG. 2 is a schematic diagram illustrating a configuration of an ultrasonic diagnostic apparatus including an ultrasonic probe according to an embodiment of the present invention;

FIGS. 3A and 3B are each an image obtained by photographing a state of an acoustic matching layer after dicing with a microscope; and

FIGS. 4A and 4B are each an image obtained by photographing states of an acoustic matching layer and a piezoelectric material after an ultrasonic probe is immersed in ethanol with a microscope.

### DETAILED DESCRIPTION OF EMBODIMENTS

Hereinafter, one or more embodiments of the present invention will be described with reference to the drawings. However, the scope of the invention is not limited to the disclosed embodiments.

1. Ultrasonic Transducer

1-1. Configuration of Ultrasonic Transducer

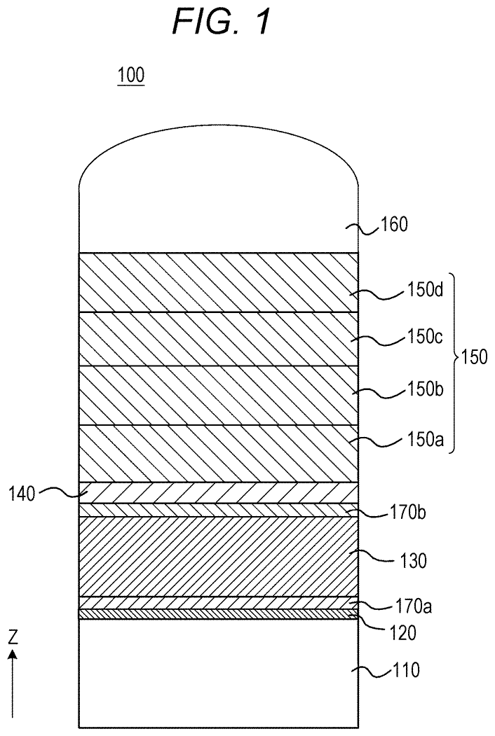

FIG. 1 is a cross-sectional view illustrating an example of an entire structure of an ultrasonic transducer 100 according to an embodiment of the present invention.

As illustrated in FIG. 1, the ultrasonic transducer 100 includes a backing material 110, a flexible printed circuit board 120, a piezoelectric material 130, an adhesive layer 140, an acoustic matching layer 150, and an acoustic lens 160. Hereinafter, each component will be described with reference to the drawings.

In the present embodiment, the “acoustic member” is a generic term for members used for the ultrasonic transducer 100, and includes the backing material 110, the flexible printed circuit board 120, the piezoelectric material 130, the acoustic matching layer 150, and the acoustic lens 160. In the present embodiment, the “ultrasonic transducer” refers to a laminate of acoustic members included in an ultrasonic probe.

In the present specification, a direction from the piezoelectric material 130 toward the acoustic lens 160 (Z direction in FIG. 1) is defined as a direction in which an ultrasonic wave is transmitted, and a generic term combining the direction in which an ultrasonic wave is transmitted and a direction opposite thereto is referred to as an ultrasonic wave propagation direction.

(Backing Material)

The backing material 110 is a member for supporting the flexible printed circuit board 120 described later, the piezoelectric material 130, and the like. The piezoelectric material 130 described later oscillates an ultrasonic wave in a direction in which an ultrasonic wave is transmitted and also slightly oscillates an ultrasonic wave in a direction opposite to the direction in which an ultrasonic wave is transmitted by volume vibration. The backing material 110 also functions as a member for attenuating an ultrasonic wave in the opposite direction emitted from the piezoelectric material 130.

In the present embodiment, the backing material 110 includes one layer, but the backing material 110 may be a laminate of a plurality of layers.

A material contained in the backing material 110 is not particularly limited. Examples of the material include an epoxy resin and a urethane resin. The backing material 110 may contain organic particles such as silicone rubber particles in order to adjust the function of attenuating an ultrasonic wave.

The thickness of the backing material 110 in the ultrasonic wave propagation direction is appropriately selected according to a material of the backing material 110, an oscillation wavelength of the ultrasonic transducer 100, and the like, but is preferably 0.5 mm or more and 10.0 mm or less, and more preferably 2.0 mm or more and 5.0 mm or less. When the thickness of the backing material 110 is in the above range, an ultrasonic wave in the opposite direction can be sufficiently attenuated. When the thickness is 0.5 mm or more, it is possible to make it difficult to reflect an ultrasonic wave from the piezoelectric material 130, and when the thickness is 10 mm or less, it is possible to downsize the backing material 110, and better workability is obtained.

(Flexible Printed Circuit Board)

The flexible printed circuit board (hereinafter, referred to as FPC) 120 functions as a member for transmitting a signal to the piezoelectric material 130 described later via signal electrodes 170a and 170b and receiving a signal from the piezoelectric material 130 via the signal electrodes 170a and 170b. In the present embodiment, the FPC 120 is disposed between the backing material 110 and the piezoelectric material 130, and is electrically connected to an external power supply, a diagnostic apparatus, and the like. Note that the FPC 120 may be disposed between the piezoelectric material 130 and the acoustic matching layer 150 (adhesive layer 140) in addition to between the backing material 110 and the piezoelectric material 130.

(Piezoelectric Material)

The piezoelectric material 130 is disposed on the FPC 120 disposed on the backing material 110 and functions as a member that transmits and receives an ultrasonic wave.

The thickness of the piezoelectric material 130 in the ultrasonic wave propagation direction is appropriately selected according to the type of an ultrasonic transducer and a frequency at which the ultrasonic transducer oscillates, but is, for example, 50 μm or more and 400 μm or less.

Examples of the piezoelectric material 130a include: a piezoelectric ceramic such as lead zirconate titanate (PZT); a piezoelectric single crystal such as lead magnesium niobate/lead titanate solid solution (PMN-PT) or lead zirconate niobate/lead titanate solid solution (PZN-PT); and a composite piezoelectric material obtained by combining these materials and a polymer material.

The plurality of signal electrodes 170a and 170b disposed on both surfaces of the piezoelectric material 130 are electrodes for applying a voltage to the piezoelectric material 130. The signal electrodes 170a and 170b are not particularly limited as long as the signal electrodes 170a and 170b are electrically connected to the above-described FPC 120 and can sufficiently exchange signals with the piezoelectric material 130, and can be layers made of, for example, gold, silver, or copper.

(Adhesive Layer)

The adhesive layer 140 includes a silane coupling agent having a structure represented by general formula (1) and an adhesive, and joins any two of the plurality of acoustic members to each other.

As described above, as a method for further improving adhesive strength between acoustic members, a method using a silane coupling agent is known. The silane coupling agent has, at both ends of a molecular chain thereof, a reactive functional group such as a mercapto group having good reactivity with an organic material such as a resin, and an alkoxy group having good reactivity with a hydroxy group present on a surface of an inorganic material such as a metal. As a result, by using the silane coupling agent, the functional group and the alkoxy group are bonded to a surface of the organic material and a surface of the inorganic material, respectively, and adhesion therebetween can be enhanced. Therefore, use of the silane coupling agent is considered to be particularly useful for improving adhesive strength between the inorganic material and the organic material.

However, as described above, even when acoustic members are bonded to each other using a coupling agent containing γ-mercaptopropyltrimethoxysilane described in JP 2003-284192 A and an adhesive, or acoustic members are bonded to each other using an adhesive containing a sulfur-containing alkoxysilane described in JP 2005-139458 A, sufficient adhesive strength cannot be obtained in some cases.

The present inventors considered that the adhesive strength would be enhanced by removing minute dirt present on surfaces of acoustic member to be bonded, and attempted to bond the acoustic members to each other after performing surface treatment such as oxygen plasma treatment on the surfaces. However, even in this case, sufficient adhesive strength could not be obtained using the silane coupling agents described in JP 2003-284192 A and JP 2005-139458 A.

Therefore, when dicing is performed at the time of manufacturing an ultrasonic transducer, one acoustic member to be bonded may be peeled off from the other acoustic member due to stress applied to the laminate of the plurality of acoustic members.

In addition, an ultrasonic probe incorporating an ultrasonic transducer is used in contact with a human body as described above, and therefore it is necessary to disinfect and clean the ultrasonic probe, for example, by immersing the ultrasonic probe in a disinfection liquid after use. When the ultrasonic probe is disinfected and cleaned, a chemical solution may infiltrate into the ultrasonic probe. When the chemical solution that has infiltrated into the ultrasonic probe enters a space between bonded acoustic members in the ultrasonic transducer, the adhesive swells. Therefore, the adhesive strength may decrease. Therefore, one of acoustic members to be bonded may be peeled off from the other acoustic member.

Therefore, the present inventors considered to improve adhesive strength between acoustic members by changing the type of the silane coupling agent to be used.

The present inventors made intensive studies, and as a result, have found that when an adhesive layer containing a silane coupling agent having a structure represented by general formula (1) and an adhesive is applied to surfaces of acoustic members and the acoustic members are bonded to each other, adhesive strength between the acoustic members is improved. Note that, in general formula (1), R₁s each independently represent a methoxy group or an ethoxy group, R₂ represents a methoxy group, an ethoxy group, or a hydrogen atom, X represents a linear or branched organic chain having 4 or more continuous carbon atoms, and A represents a reactive functional group.

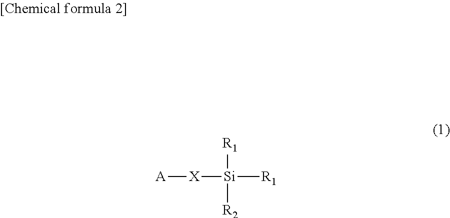

Regarding these results, according to study of the present inventors, in each of the γ-mercaptopropyltrimethoxysilane described in JP 2003-284192 A and the sulfur-containing alkoxysilane described in JP 2005-139458 A, an organic chain bonded between the Si atom of the silane coupling agent and the reactive functional group has 3 or less atoms (carbon atoms). Therefore, it is considered that it is difficult to sufficiently enhance an orientation property at an adhesive surface. Therefore, it is considered that since molecules cannot be oriented at a sufficient density on an adhesion surface between the acoustic members, sufficient adhesive strength cannot be obtained.

On the other hand, in the silane coupling agent used in the present invention, since an organic chain in a molecular structure thereof has continuous 4 or more carbon atoms, it is considered that the silane coupling agent can be oriented on an adhesive surface between acoustic members with a sufficiently high density, and the adhesive strength can be improved.

Then, the present inventors have found that by using the silane coupling agent, adhesive strength sufficient for suppressing peeling of an acoustic member that occurs at the time of dicing or disinfection and cleaning can be obtained.

In the present specification, the “organic chain” refers to a moiety other than a silane alkoxide group and a reactive functional group in the silane coupling agent, the moiety containing a carbon atom and having a straight chain or a branched chain.

As described above, the organic chain has 4 or more continuous carbon atoms. The number of carbon atoms is preferably 4 or more and 12 or less, and more preferably 4 or more and 6 or less. When the organic chain has 4 or more carbon atoms, interaction between the organic chains can be enhanced, the molecules can be oriented at a sufficient density, and adhesive strength between acoustic members can be further enhanced. When the silane coupling agent is contained in the adhesive layer, a solution in which the silane coupling agent is dissolved may be used. At this time, in the molecular structure of the silane coupling agent, when the organic chain has 12 or less carbon atoms, solubility in the solution containing the silane coupling agent can be enhanced, and the silane coupling agent can be easily contained in the adhesive layer.

The organic chain may include a structure such as —O—, —(NH)—, or —S— in a straight chain or a branched chain.

The silane coupling agent has a silane alkoxide group (—Si(R₁)₂(R₂)) in general formula (1)) having a structure in which a plurality of alkoxy groups is bonded to a Si atom. In general formula (1), R₁s each independently represent a methoxy group or an ethoxy group, and R₂ represents a methoxy group, an ethoxy group, or a hydrogen atom. The number of alkoxy groups bonded to one Si atom is preferably three from a viewpoint of sufficiently bonding the silane coupling agent to an acoustic member containing an inorganic material to further enhance adhesive strength. Therefore, R₂ is preferably a methoxy group or an ethoxy group.

The type of the reactive functional group (A in general formula (1)) contained in the silane coupling agent is not particularly limited, and examples thereof include a mercapto group, a vinyl group, an acryloyl group, an epoxy group, an amino group, and an isocyanate group. Among these groups, the functional group is preferably a mercapto group, a vinyl group, an acryloyl group, an epoxy group, an amino group, or an isocyanate group from a viewpoint of further enhancing adhesive strength between acoustic members. In addition, when an acoustic member containing a resin is bonded to another acoustic member, the functional group is preferably an amino group. For example, when a piezoelectric material including an electrode made of gold is bonded to an acoustic matching layer, the functional group is preferably a mercapto group.

The weight average molecular weight (Mw) of the silane coupling agent is not particularly limited, but is preferably 200 or more and 400 or less. When the molecular weight is 200 or more, the number of atoms (carbon number) of the organic chain of the silane coupling agent increases, interaction between the organic chains can be enhanced, and the silane coupling agent can be oriented at a sufficient density. Therefore, the adhesive strength can be further improved. When the molecular weight is 400 or less, solubility in the solution containing the silane coupling agent can be enhanced, and the silane coupling agent can be easily contained in the adhesive layer. The molecular weight may be obtained by performing measurement by gel permeation chromatography (GPC) using polystyrene as a standard

As the silane coupling agent, a commercially available product may be used. Examples of the commercially available product include KBM-1083, KBM-4803, KBM-5803, and KBM-6803 (all of which are manufactured by Shin-Etsu Chemical Co., Ltd.), and SIM6480.0, SIA0587.0, SIA0592.0, and SIA0630.0 (all of which are manufactured by AZmax. co).

A position where the adhesive layer 140 is disposed is not particularly limited as long as the adhesive layer 140 is disposed in at least one of spaces between the plurality of acoustic members. In the present embodiment, the adhesive layer 140 is disposed between the piezoelectric material 130 and the acoustic matching layer 150 and joins the piezoelectric material 130 and the acoustic matching layer 150 to each other.

The adhesive layer 140 preferably contains an organic acid having 2 or more and 6 or less carbon atoms or a salt thereof.

The alkoxy group contained in the silane alkoxide group of the silane coupling agent generates a hydroxy group by hydrolysis that occurs in the solution containing the silane coupling agent. The hydroxy group reacts with a hydroxy group present on a surface of an inorganic material, whereby the silane coupling agent and the surface of the inorganic material are bonded to each other. At this time, since the adhesive layer 140 contains an organic acid, hydrolysis can be promoted under acidic conditions. Therefore, a hydroxy group is easily generated from the alkoxy group. Therefore, reactivity between the silane coupling agent and the hydroxy group present on the surface of the inorganic material can be increased to sufficiently bond the silane coupling agent and the surface of the inorganic material to each other, and the adhesive strength between acoustic members can be further enhanced. For this reason, the organic acid is preferably contained in the solution containing the silane coupling agent, and the organic acid is preferably contained in the adhesive layer 140 by applying the solution to a surface of an acoustic member.

Examples of the organic acid include acetic acid, propionic acid, pentanoic acid, butyric acid, hexanoic acid, citric acid, and lactic acid.

When the organic acid has 2 or more carbon atoms, reactivity between the silane coupling agent and a hydroxy group present on the surface of the inorganic material can be enhanced to sufficiently bond the silane coupling agent and the surface of the inorganic material to each other, and the adhesive strength between acoustic members can be further enhanced. When the organic acid has 6 or less carbon atoms, the organic acid is easily dissolved in the solution containing the silane coupling agent. The organic acid preferably has 2 or more and 6 or less carbon atoms from the above viewpoint.

When acoustic members are bonded to each other, an adhesive contained in the adhesive layer may be heated to moderately lower viscosity thereof. At this time, since the organic acid having 2 or more and 6 or less carbon atoms has a boiling point of 60° C. or higher, volatilization of the organic acid from the adhesive layer 140 can be suppressed. The boiling point of the organic acid is preferably 60° C. or higher and 210° C. or lower, and more preferably 100° C. or higher and 180° C. or lower from the above viewpoint.

The type of the adhesive contained in the adhesive layer 140 is not particularly limited, but is, for example, an epoxy-based adhesive containing an epoxy resin or a silicone adhesive containing a silicone resin.

The adhesive has a glass transition temperature (T_{g}) of 60° C. or higher. As a result, it is possible to suppress softening of the adhesive by frictional heat generated when a bonded acoustic member is diced. Therefore, when the glass transition temperature (T_{g}) is 60° C. or higher, it is possible to further suppress peeling of an acoustic member at the time of dicing. The glass transition temperature (T_{g}) of the adhesive is preferably 60° C. or higher and 200° C. or lower from the above viewpoint. When the glass transition temperature (T_{g}) is 200° C. or lower, a heating amount required for applying the adhesive can be suppressed. The glass transition temperature (T_{g}) can be measured using a differential scanning calorimeter “Diamond DSC” (manufactured by PerkinElmer, Inc.) under temperature raising and cooling conditions in which a temperature raising/lowering rate is 10° C./min and a temperature raising range is from 0° C. to 150° C.

The thickness of the adhesive layer 140 in the ultrasonic wave propagation direction is not particularly limited, but is preferably 1 μm or less, and more preferably 0.1 μm or more and less than 1 μm. When the thickness of the adhesive layer 140 is 1 μm or less, reflection of an ultrasonic wave due to a difference in acoustic impedance between acoustic members to be bonded can be more sufficiently suppressed. When the thickness of the adhesive layer 140 is 0.1 μm or more, the adhesive strength can be more sufficiently enhanced.

A surface of an acoustic member on which the adhesive layer 140 is disposed is preferably subjected to surface treatment such as alkaline acid cleaning, UV treatment, or oxygen plasma treatment. As a result, it is possible to remove minute dirt present on the surface of the acoustic member. Therefore, it is possible to suppress a decrease in adhesive strength due to the dirt.

Note that the adhesive layer 140 may be disposed in any one of spaces between the acoustic members (the backing material 110, the FPC 120, the piezoelectric material 130, the acoustic matching layer 150, and the acoustic lens 160 in the present embodiment), may be disposed in any two of spaces between the acoustic members, or may be disposed in each of spaces between the acoustic members.

(Acoustic Matching Layer)

The acoustic matching layer 150 is a layer disposed on the piezoelectric material 130 disposed on the FPC 120, and functions as a member for adjusting an acoustic impedance between the piezoelectric material 130 and the acoustic lens 160. The acoustic matching layer 150 may include one layer, or may include a plurality of layers having different acoustic impedances. The number of layers of the acoustic matching layer 150 is not particularly limited, and is generally two or more. As illustrated in FIG. 1, in the present embodiment, the acoustic matching layer 150 is a laminate including a first matching layer 150a, a second matching layer 150b, a third matching layer 150c, and a fourth matching layer 150d.

The acoustic matching layer 150 preferably contains a resin. That is, at least one of the acoustic matching layers 150a, 150b, 150c, and 150d preferably contains a resin from a viewpoint of easily adjusting the acoustic impedance of the acoustic matching layer 150. Examples of the resin contained in the acoustic matching layer 150 include an epoxy resin, a urethane resin, a silicone resin, and a polystyrene resin. In addition, the acoustic matching layer 150 may contain a curing agent that cures these resins.

The acoustic matching layer 150 may contain inorganic particles. A material of the inorganic particles contained in the acoustic matching layer 150 is not particularly limited, and examples thereof include ferrite, silicone rubber, tungsten oxide, and tungsten.

As described above, when the acoustic matching layer 150 contains a resin, the acoustic impedance of the acoustic matching layer 150 is easily adjusted. On the other hand, when a method for forming an acoustic matching layer by applying a resin composition constituting the acoustic matching layer on the acoustic matching layer and then curing the resin composition is used, the resin shrinks during curing. As a result, the acoustic matching layer 150 may be bent, and may be peeled off from a bonded piezoelectric material or another bonded acoustic matching layer. This phenomenon is particularly likely to occur when the content of the inorganic particles in the acoustic matching layer 150 is small.

When the ultrasonic probe is disinfected and cleaned with a chemical solution, the chemical solution may enter the probe and swell the acoustic matching layer 150. As a result, the swollen acoustic matching layer 150 is may be bent, and may be peeled off from a bonded piezoelectric material or another bonded acoustic matching layer. This phenomenon is particularly likely to occur when the resin in the acoustic matching layer 150 has a low crosslinking density.

On the other hand, in the present embodiment, since the piezoelectric material 130 (the electrode 170b on the piezoelectric material 130 in the present embodiment) and the acoustic matching layer 150 are bonded to each other via the adhesive layer 140, the adhesive strength between the piezoelectric material 130 and the acoustic matching layer 150 can be enhanced to suppress peeling due to bending of the acoustic matching layer 150.

The adhesive layer 140 is preferably disposed in any one of spaces between the first matching layer 150a, the second matching layer 150b, the third matching layer 150c, and the fourth matching layer 150d described later, more preferably disposed in any two of spaces between these layers, and still more preferably disposed in each of spaces between these layers from a viewpoint of suppressing peeling that occurs between the acoustic matching layers 150a to 150d due to bending of the acoustic matching layer 150 by shrinkage during curing or swelling during disinfection and cleaning described above.

By disposing the adhesive layer 140 between the acoustic matching layers 150a to 150d, even when the acoustic matching layers cured in advance are laminated with and bonded to each other, the adhesive strength between the acoustic matching layers can be sufficiently enhanced. This is because when at least one of the acoustic matching layers 150 to be bonded contains inorganic particles, the silane alkoxide group of the silane coupling agent reacts with the inorganic particles, and the reactive functional group forms a bond with the resin contained in the other acoustic matching layer.

The acoustic matching layer 150 preferably includes a layer containing a resin and particles having a specific gravity of 4.5 or more and 6.0 or less from a viewpoint of easily adjusting the acoustic impedance of the acoustic matching layer 150. By including a layer containing such particles, it is possible to suppress a decrease in sound speed in the acoustic matching layer 150 and to suppress an excessive decrease in acoustic impedance while increasing the density of the acoustic matching layer 150. Examples of the particles having a specific gravity in the above range include ferrite and zinc oxide. These particles may be used singly or in combination of two or more types thereof.

The specific gravity of the particles contained in the acoustic matching layer is more preferably 4.5 or more and 5.6 or less from a viewpoint of easily adjusting the acoustic impedance of the acoustic matching layer 150.

When an ultrasonic wave is propagated between different media, the ultrasonic wave is reflected in proportion to the magnitude of a difference in acoustic impedance between the media. Therefore, it is preferable to adjust the acoustic impedance of the acoustic matching layer 150 such that a difference between the acoustic impedance (about 29 to 35 MRayls) of the piezoelectric material 130 and the acoustic impedance (about 1.53 MRayls) of a living body to be brought into contact with the ultrasonic probe described later is gradually reduced from the piezoelectric material 130 toward the acoustic lens 160. As described above, the particles having a specific gravity in the above range can suppress an excessive decrease in the acoustic impedance of the acoustic matching layer 150, and are therefore preferably included in an acoustic matching layer (the acoustic matching layer 150a in the present embodiment) located closest to the piezoelectric material in the acoustic matching layer 150. As a result, the difference between the acoustic impedance of the acoustic matching layer 150 and the acoustic impedance of the living body can be gradually reduced from the piezoelectric material 130 toward the acoustic lens 160.

The acoustic impedance of the acoustic matching layer 150a disposed closest to the piezoelectric material in the acoustic matching layer 150 is preferably 10 MRayls or more and 25 MRayls or less, and more preferably adjusted to 11 MRayls or more and 15 MRayls or less by containing particles having a specific gravity in the above range from the above viewpoint.

The content of the particles having a specific gravity of 4.5 or more and 6.0 or less is preferably 150% by mass or more and 1200% by mass or less with respect to the total mass of the resin in the layer containing the resin and the particles. In the present embodiment, at least one of the acoustic matching layers 150a, 150b, 150c, and 150d preferably contains the particles in an amount of 150% by mass or more and 1200% by mass or less with respect to the mass of the resin contained in one layer. When the content is 150% by mass or more, the density of each of the acoustic matching layers 150a to 150d can be increased to increase the acoustic impedance. When the content is 1200% by mass or less, it is possible to suppress a decrease in sound speed in each of the acoustic matching layers 150a to 150d and to suppress an excessive decrease in the acoustic impedance. Among the particles, for example, the content of particles having a specific gravity of 4.5 is preferably 160% by mass or more and 880% by mass or less, and the content of particles having a specific gravity of 6.0 is preferably 215% by mass or more and 1165% by mass or less. Note that, in the present embodiment, the “mass of the resin” represents the total mass of the resin and the curing agent.

The acoustic matching layer 150 preferably includes a layer containing elastomer particles. Since the elastomer particles tend to have a smaller specific gravity than the inorganic particles, inclusion of the elastomer particles can reduce the sound speed and the density in the acoustic matching layer 150 to suppress an excessive increase in acoustic impedance. Among the acoustic matching layers 150a, 150b, 150c, and 150d, a matching layer disposed at a position farthest from the piezoelectric material 130 (the fourth matching layer 150d in the present embodiment) in the ultrasonic wave transmission direction preferably contains the elastomer particles from such a viewpoint. As a result, it is possible to facilitate adjustment so as to approach the acoustic impedance of a living body to be brought into contact with the ultrasonic probe.

The content of the elastomer particles is preferably 4 parts by mass or more and 122 parts by mass or less, and more preferably 9 parts by mass or more and 100 parts by mass or less with respect to 100 parts by mass of the resin in the layer containing the elastomer particles. The content of the elastomer particles contained in the matching layer disposed at a position farthest from the piezoelectric material 130 (the fourth matching layer 150d in the present embodiment) is preferably 27 parts by mass or more and 100 parts by mass or less, and more preferably 54 parts by mass or more and 81 parts by mass or less with respect to 100 parts by mass of the resin of the matching layer disposed at a position farthest from the piezoelectric material 130.

The acoustic impedance of each of the layers constituting the acoustic matching layer 150 can be appropriately adjusted by changing the type and amount of a component constituting each of the layers. For example, when each of the matching layers 150a, 150b, 150c, and 150d contains a resin and particles, the acoustic impedance can be adjusted by changing the type and amount of the particles in each of the matching layers. Note that the acoustic matching layers 150a, 150b, 150c, and 150d may contain the same resin and particles, or may contain different resins and particles. Furthermore, the thicknesses of the layers may be the same or different.

The thickness of each of the acoustic matching layers 150a, 150b, 150c, and 150d in the ultrasonic wave propagation direction is not particularly limited, but is preferably appropriately adjusted according to the wavelength of an ultrasonic wave to be used from a viewpoint of suppressing reflection of the ultrasonic wave due to a difference in acoustic impedance from the acoustic lens. For example, when an ultrasonic wave having a center frequency of 10 MHz is used, the thickness is preferably 20 μm or more and 80 μm or less. The thickness is more preferably substantially equal to ¼ of the wavelength of the ultrasonic wave from the above viewpoint. The “substantially equal thickness” refers to a thickness of 95% or more and 105% or less with respect to the thickness of ¼ of the wavelength of the ultrasonic wave.

(Acoustic Lens)

The acoustic lens 160 is a member for focusing an ultrasonic wave transmitted from the piezoelectric material 130. As illustrated in FIG. 1, in the present embodiment, the acoustic lens 160 is a cylindrical acoustic lens extending in the Y direction in FIG. 1 and protruding in the Z direction. The shapes of cross sections perpendicular to the X direction are all the same. The acoustic lens 160 focuses an ultrasonic wave oscillated by the piezoelectric material 130 in the Z direction and emits the ultrasonic wave to the outside of the ultrasonic transducer 100.

The acoustic lens 160 is made of a material having acoustic characteristics suitable for an object to be inspected, for example, a living body. For example, the acoustic lens 160 is preferably made of a material having an acoustic impedance relatively close to an acoustic impedance of an object to be inspected, such as silicone rubber.

1-2. Method for Manufacturing Ultrasonic Transducer

Hereinafter, a method for manufacturing an ultrasonic transducer capable of manufacturing the above-described ultrasonic transducer 100 will be described.

A method for manufacturing an ultrasonic transducer in the present embodiment includes: a step of disposing an adhesive layer containing a silane coupling agent having a structure represented by the above general formula (1) and an adhesive on a surface of at least one acoustic member among a plurality of acoustic members; and a step of laminating another acoustic member on the surface on which the adhesive layer is disposed.

(Step of Disposing Adhesive Layer)

In this step, the adhesive layer 140 containing the silane coupling agent and an adhesive is disposed on a surface of at least one acoustic member among a plurality of acoustic members.

A method for disposing the adhesive layer 140 on a surface of an acoustic member is not particularly limited. For example, the adhesive layer 140 can be disposed by immersing an acoustic member in a solution containing a polyfunctional silane coupling agent and then applying an adhesive to the acoustic member.

The content of the silane coupling agent in the solution containing the silane coupling agent is preferably 1% by mass or more and 15% by mass or less, and more preferably 1% by mass or more and 10% by mass or less with respect to the total mass of the solution.

In the method for disposing the adhesive layer 140 on a surface of an acoustic member, the adhesive layer 140 is preferably disposed by disposing an adhesive containing the silane coupling agent on a surface of an acoustic member from a viewpoint of more sufficiently enhancing adhesive strength between acoustic members. The silane coupling agent has low compatibility with the adhesive, and has high reactivity with an inorganic material and an organic material contained in an acoustic member as described above. Therefore, when the silane coupling agent is contained in the adhesive, the silane coupling agent easily gathers on a surface side of an acoustic member to be bonded in the adhesive layer, and the concentration of the coupling agent in the adhesive layer increases as it goes toward an adhesive surface. As a result, the density of the silane coupling agent on the adhesive surface can be increased to enhance the adhesive strength. In addition, it is possible to sufficiently suppress multilayering of the silane coupling agent as compared with a case of directly applying the silane coupling agent to the adhesive surface, and inhibition of bonding between the silane coupling agent and the adhesive can be sufficiently suppressed to improve the adhesive strength.

The acoustic member on which the adhesive layer 140 is disposed is not particularly limited as long as another acoustic member is laminated on the acoustic member.

(Step of Laminating Acoustic Member)

In this step, on the surface of the acoustic member on which the adhesive layer 140 is disposed, another acoustic member is laminated.

On the surface of the acoustic member on which the adhesive layer 140 is disposed, another acoustic member is laminated, whereby these acoustic members can be bonded to each other. When an acoustic member is laminated, pressure may be applied to the acoustic member to pressure-bond the acoustic member as necessary.

(Step of Performing Oxygen Plasma Treatment)

In the present embodiment, the method for manufacturing an ultrasonic transducer may include a step of subjecting a surface of an acoustic member to oxygen plasma treatment.

In this step, by subjecting a surface of an acoustic member to oxygen plasma treatment, it is possible to remove minute dirt present on the surface of the acoustic member. As a result, it is possible to suppress a decrease in adhesive strength between acoustic members due to the dirt. Therefore, this step is performed before the step of disposing the adhesive layer 140 on a surface of an acoustic member.

When the oxygen plasma treatment is performed, a flow rate of an oxygen gas is not particularly limited, but is, for example, 1 sccm or more and 100 sccm or less. Time during which the oxygen plasma treatment is performed is not particularly limited, but is, for example, 30 seconds or more and 300 seconds or less.

(Dicing Step)

In the present embodiment, the method for manufacturing an ultrasonic transducer may include a step of dicing a laminate in which a plurality of acoustic members is laminated.

In this step, a laminate in which a plurality of acoustic members is laminated is diced. As a result, the ultrasonic transducer can be cut into a size according to a use application.

2. Ultrasonic Probe and Ultrasonic Diagnostic Apparatus

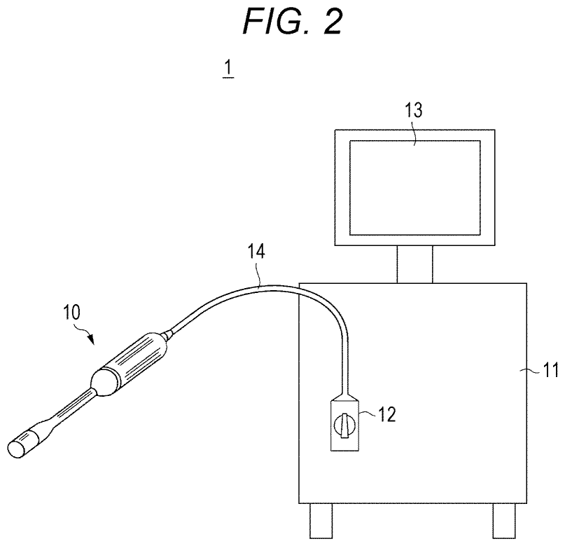

The above-described ultrasonic transducer can be used for, for example, an ultrasonic probe 10, an ultrasonic diagnostic apparatus 1, and the like as illustrated in FIG. 2. The ultrasonic diagnostic apparatus 1 includes the ultrasonic probe 10 including the above-described ultrasonic transducer 100, a main body 11, a connector 12, a display 13, and the like.

The ultrasonic probe 10 only needs to include the ultrasonic transducer (not illustrated), and is connected to the main body 11 via a cable 14 connected to the connector 12.

An electric signal (transmission signal) from the main body 11 is transmitted to a piezoelectric material of the ultrasonic probe 10 via the cable 14. The transmission signal is converted into an ultrasonic wave by the piezoelectric material and transmitted into an object to be inspected. The transmitted ultrasonic wave is reflected in the object to be inspected. Then, a part of the reflected wave is received by the piezoelectric material, converted into an electric signal (reception signal), and transmitted to the main body 11. The reception signal is converted into image data in the main body 11 of the ultrasonic diagnostic apparatus 1 and displayed on the display 13.

### EXAMPLES

Hereinafter, the present invention will be specifically described with reference to Examples, but the present invention is not limited thereto.

1. Adhesive Strength Measurement Test

(Preparation of Surface Treatment Liquid)

2 parts by mass of a silane coupling agent 1 (SIM6480.0, manufactured by AZmax. co) was diluted with 66 parts by mass of methanol and 32 parts by mass of water to prepare a surface treatment liquid 1a. Surface treatment liquids 1b to 6b were prepared in a similar manner except that the silane coupling agent to be used and the solvent for dilution were changed as presented in Table 1.

(Preparation of Adhesive Liquids A1 to F and Adhesive Liquid 0)

A main agent and a curing agent of an adhesive (C1163, manufactured by Tesk Co., Ltd., glass transition temperature: 55° C.) were mixed at 2:1, and the silane coupling agent 1 was further added thereto such that the content of the silane coupling agent 1 was 2 wt % with respect to the total mass of the adhesive. The mixture of the adhesive and the silane coupling agent 1 was sufficiently mixed in a vacuum mixer (ARV-310P, manufactured by THINKY CORPORATION) at a rotation speed of 2000 rpm and 0.5 kPa for one minute to prepare an adhesive liquid A1.

Adhesive liquids A2 to F and an adhesive liquid 0 were prepared in a similar manner to the adhesive liquid A1 except that the type and mass of the silane coupling agent added to the adhesive were changed as presented in Table 2.

Note that, as silane coupling agents 1 to 8 in Tables 1 and 2, those described below were used.

Silane coupling agent 1 (SIM6480.0, manufactured by AZmax. co)

Silane coupling agent 2 (M0298, manufactured by Shin-Etsu Chemical Co., Ltd.)

Silane coupling agent 3 (SIA0587.0, manufactured by AZmax. co)

Silane coupling agent 4 (SIA0592.6, manufactured by AZmax. co)

Silane coupling agent 5 (SIA0630.0, manufactured by AZmax. co)

Silane coupling agent 6 (KBM-903, manufactured by Shin-Etsu Chemical Co., Ltd.)

Silane coupling agent 7 (KBM-6803, manufactured by Shin-Etsu Chemical Co., Ltd.)

Silane coupling agent 8 (KBM-4803, manufactured by Shin-Etsu Chemical Co., Ltd.)

The structures of the silane coupling agents 1 to 8 are as follows. Note that the numbers 1 to 8 assigned to the structural formulas indicated below correspond to the silane coupling agent 1 to 8, respectively.

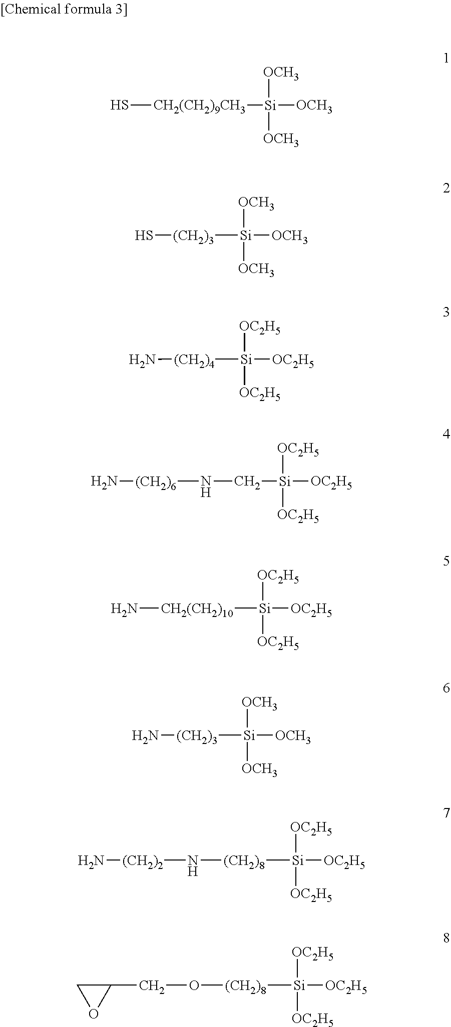

TABLE 1

| | Silane coupling agent | Solvent | |
|---|---|---|---|
| Surface treatment liquid 1a | Silane coupling agent 1 | Methanol | Water |
| | 2 parts by mass | 66 parts by mass | 32 parts by mass |
| Surface treatment liquid 1b | Silane coupling agent 1 | Methanol | 1 wt % acetic acid aqueous solution |
| | 2 parts by mass | 66 parts by mass | 32 parts by mass |
| Surface treatment liquid 2a | Silane coupling agent 2 | Methanol | Water |
| | 1 part by mass | 66 parts by mass | 33 parts by mass |
| Surface treatment liquid 2b | Silane coupling agent 2 | Ethanol | 1 wt % acetic acid aqueous solution |
| | 1 part by mass | 66 parts by mass | 33 parts by mass |
| Surface treatment liquid 2c | Silane coupling agent 2 | Ethanol | 1 wt % acetic acid aqueous solution |
| | 2 parts by mass | 66 parts by mass | 32 parts by mass |
| Surface treatment liquid 3a | Silane coupling agent 3 | Methanol | Water |
| | 2 parts by mass | 66 parts by mass | 32 parts by mass |
| Surface treatment liquid 3b | Silane coupling agent 3 | Methanol | 1 wt % acetic acid aqueous solution |
| | 2 parts by mass | 66 parts by mass | 32 parts by mass |
| Surface treatment liquid 4 | Silane coupling agent 4 | Methanol | Water |
| | 2 parts by mass | 66 parts by mass | 32 parts by mass |
| Surface treatment liquid 5 | Silane coupling agent 5 | Methanol | Water |
| | 2 parts by mass | 66 parts by mass | 32 parts by mass |
| Surface treatment liquid 6a | Silane coupling agent 6 | Methanol | Water |
| | 1 part by mass | 66 parts by mass | 33 parts by mass |
| Surface treatment liquid 6b | Silane coupling agent 6 | Methanol | 1 wt % acetic acid aqueous solution |
| | 2 parts by mass | 66 parts by mass | 32 parts by mass |

TABLE 2

| No. | Silane coupling agent |
|---|---|
| Adhesive liquid A1 | Silane coupling agent 1 |
| | 2 parts by mass |
| Adhesive liquid A2 | Silane coupling agent 1 |
| | 1 part by mass |
| Adhesive liquid B | Silane coupling agent 2 |
| | 2 parts by mass |
| Adhesive liquid C | Silane coupling agent 5 |
| | 2 parts by mass |
| Adhesive liquid D | Silane coupling agent 7 |
| | 2 parts by mass |
| Adhesive liquid E | Silane coupling agent 8 |
| | 2 parts by mass |
| Adhesive liquid F | Silane coupling agent 6 |
| | 2 parts by mass |
| Adhesive liquid 0 | — |

(Preparation of Acoustic Matching Layer Substrate)

90 parts by mass of an epoxy resin (jER-630, manufactured by Mitsubishi Chemical Corporation), 5 parts by mass of an epoxy resin curing agent (jER Cure WA, manufactured by Mitsubishi Chemical Corporation), 5 parts by mass of an epoxy resin curing agent (CUREZOL 1B2MZ, manufactured by Shikoku Chemicals Corporation), 373 parts by mass of ferrite powder (LD-M, manufactured by JFE Chemical Corporation), and 322 parts by mass of tungsten powder (W-2KD, manufactured by JAPAN NEW METALS CO., LTD.) were sufficiently mixed using a vacuum mixer (ARV-310P, manufactured by THINKY CORPORATION) at a rotation speed of 2000 rpm and a vacuum pressure of 0.5 kPa for five minutes to prepare a compound-1.

A glass substrate was cleaned with a neutral detergent, then sufficiently cleaned with pure water, and dried. The resulting glass substrate was immersed in a surface treatment liquid 4 for five minutes, and then dried in a thermostatic chamber at 60° C. for 20 minutes. Thereafter, the glass substrate was cleaned with pure water for three minutes, and dried again in a thermostatic chamber at 60° C. for five minutes to prepare a surface-treated glass substrate.

The glass substrate and a blade were heated to 75° C. Thereafter, the compound-1 at 75° C. was applied onto the glass substrate so as to have a thickness of 100 μm, dried in a thermostatic chamber at 80° C. for one hour, and then further heated in a thermostatic chamber at 150° C. for three hours to prepare an acoustic matching layer substrate.

(Preparation of Adhesion Test Samples 1 to 12)

An adhesive film (UPICEL N SE1420, manufactured by Ube Corporation) was subjected to electroless nickel plating. Subsequently, a surface of the adhesive film was plated with gold by electroplating. In this way, an electrode film simulating an electrode of a piezoelectric material was prepared. The prepared electrode film was cleaned with a neutral detergent, then sufficiently cleaned with pure water, and dried at room temperature (25° C.). Subsequently, the electrode film was immersed in the surface treatment liquid 1a for five minutes, and then dried in a thermostatic chamber at 60° C. for 20 minutes to apply a coupling agent to a surface of the gold plated film. Furthermore, the electrode film was cleaned with pure water for three minutes, and dried again in a thermostatic chamber at 60° C. for five minutes.

Subsequently, the adhesive liquid 0 was applied to a surface of the acoustic matching layer substrate on which a matching layer was formed and the gold surface of the electrode film to which the coupling agent was applied to form an adhesive layer. Thereafter, the surface of the acoustic matching layer to which the adhesive liquid 0 was applied and the surface of the electrode film to which the adhesive liquid 0 was applied were stuck to each other, and bonded to each other by applying a pressure of 30 kgf/cm at a temperature of 60° C. for three hours using a pressing jig with a spring, thereby preparing an adhesion test sample 1. At this time, the adhesive layer had a thickness of 0.8 μm. The thickness of the adhesive was measured by observing a cross section of the adhesion test sample at an acceleration voltage of 200 kV and a magnification of 200 times using an electron microscope (S-800, manufactured by Hitachi High-Tech Co., Ltd.).

An adhesion test sample 2 was prepared in a similar manner to the adhesion test sample 1 except that the surface treatment liquid in which the electrode film was immersed was changed to a surface treatment liquid 1b. An adhesion test sample 3 was prepared in a similar manner to the adhesion test sample 2 except that a surface of the electrode film was subjected to oxygen plasma treatment and then immersed in the surface treatment liquid 1b.

The oxygen plasma treatment was performed for 45 seconds using a plasma cleaner (PC-1100, manufactured by Samco Inc.) at an oxygen gas flow rate of 5 sccm and a power of 50 W.

Adhesion test samples 4 to 6 were prepared in a similar manner to the adhesion test sample 1 except that whether or not a surface of the electrode film was subjected to oxygen plasma treatment and the surface was immersed in each of the surface treatment liquids presented in Table 3.

Adhesion test samples 7 to 12 were prepared in a similar manner to the adhesion test sample 1 except that a polystyrene film was used instead of the electrode film, whether or not a surface of the polystyrene film was subjected to oxygen plasma treatment, and the surface treatment liquid used was changed as presented in Table 3.

(Preparation of Adhesion Test Samples 13 to 21)

An adhesive film (UPICEL N SE1420, manufactured by Ube Corporation) was subjected to electroless nickel plating. Subsequently, a surface of the adhesive film was plated with gold by electroplating. The electrode film prepared in this way was cleaned with a neutral detergent, then sufficiently cleaned with pure water, and dried at room temperature (25° C.).

The adhesive liquid A1 was applied to the surface of the acoustic matching layer substrate on which the matching layer was formed and the gold surface of the electrode film, the surfaces to which the adhesive liquid A1 was applied were stuck to each other, and bonded to each other by applying a pressure of 30 kgf/cm at a temperature of 60° C. for three hours using a pressing jig with a spring, thereby preparing an adhesion test sample 13. At this time, the adhesive layer had a thickness of 0.6 μm. The thickness of the adhesive was measured by observing a cross section of the adhesion test sample at an acceleration voltage of 200 kV and a magnification of 200 times using an electron microscope (S-800, manufactured by Hitachi High-Tech Co., Ltd.).

An adhesion test sample 14 was prepared in a similar manner to the adhesion test sample 13 except that a surface of the electrode film was subjected to oxygen plasma treatment and then the adhesive liquid Al was applied to the surface. The oxygen plasma treatment was performed for 45 seconds using a plasma cleaner (PC-1100, manufactured by Samco Inc.) at an oxygen gas flow rate of 5 sccm and a power of 50 W.

Adhesion test samples 15 and 16 were prepared in a similar manner to the adhesion test sample 13 except that whether or not a surface of the electrode film was subjected to oxygen plasma treatment and the adhesive liquids presented in Table 4 were used.

Adhesion test samples 17 to 21 were prepared in a similar manner to the adhesion test sample 1 except that a polystyrene film was used instead of the electrode film, whether or not a surface of the polystyrene film was subjected to oxygen plasma treatment, and the adhesive liquid used was changed as presented in Table 4. Note that bonding between the polystyrene film and the surface of the acoustic matching layer substrate on which the matching layer is formed simulates bonding between an acoustic matching layer containing polystyrene and an acoustic matching layer containing an epoxy resin.

(Measurement of Adhesive Strength)

In order to measure the adhesive strength of the prepared adhesion test samples 1 to 21, a 90 degree peeling test was performed under a temperature condition of 50° C. using a digital force gauge (ZP-20N, manufactured by IMADA CO., LTD.) and a measuring stand (MX-500N, manufactured by IMADA CO., LTD.) in accordance with a method described in JIS K6854-1: 1999. At this time, assuming that the width of each of the adhesion test samples in a direction orthogonal to a pulling direction was 1 cm, peel strength when the sample was peeled off from the acoustic matching layer was measured as the adhesive strength. Adhesiveness was evaluated according to the following criteria based on measurement results.

∘ Adhesive strength is 1.2 kgf/cm or more

Δ Adhesive strength is 1.0 kgf/cm or more and 1.2 kgf/cm or less

× Adhesive strength is 1.0 kgf/cm or less

Evaluation results are presented in Tables 3 and 4.

TABLE 3

| | | | Surface | | Thickness of |
|---|---|---|---|---|---|
| Adhesion test | | | treatment | | adhesive layer |
| sample No. | Adhesive film | Surface treatment | liquid No. | Adhesiveness | [μm] |
| 1 | Electrode film | — | 1a | ∘ | 0.8 |
| 2 | Electrode film | — | 1b | ∘ | 0.7 |
| 3 | Electrode film | Oxygen plasma | 1b | ∘ | 0.6 |
| 4 | Electrode film | Oxygen plasma | 2a | x | 1.1 |
| 5 | Electrode film | — | 2b | x | 1.1 |
| 6 | Electrode film | Oxygen plasma | 2c | Δ | 1.1 |
| 7 | Polystyrene film | Oxygen plasma | 3a | ∘ | 0.9 |
| 8 | Polystyrene film | Oxygen plasma | 3b | ∘ | 0.5 |
| 9 | Polystyrene film | Oxygen plasma | 4 | ∘ | 0.9 |
| 10 | Polystyrene film | Oxygen plasma | 5 | ∘ | 0.5 |
| 11 | Polystyrene film | — | 6a | x | 1.2 |
| 12 | Polystyrene film | Oxygen plasma | 6b | Δ | 0.9 |

TABLE 4

| | | | | | Thickness of |
|---|---|---|---|---|---|
| Adhesion test | | | Adhesive | | adhesive layer |
| sample No. | Adhesive film | Surface treatment | liquid No. | Adhesiveness | [μm] |
| 13 | Electrode film | — | A1 | ∘ | 0.6 |
| 14 | Electrode film | Oxygen plasma | A1 | ∘ | 0.4 |
| 15 | Electrode film | — | A2 | ∘ | 0.4 |
| 16 | Electrode film | — | B | Δ | 1.0 |
| 17 | Polystyrene film | — | C | ∘ | 0.5 |
| 18 | Polystyrene film | — | D | ∘ | 0.5 |
| 19 | Polystyrene film | Oxygen plasma | D | ∘ | 0.3 |
| 20 | Polystyrene film | — | E | ∘ | 0.5 |
| 21 | Polystyrene film | — | F | Δ | 0.9 |

2. Manufacture and Durability Test of Ultrasonic Transducer

An ultrasonic transducer having a configuration similar to that of the ultrasonic transducer 100 according to the present embodiment was manufactured by the following procedure. Then, a test for confirming whether or not an acoustic matching layer was peeled (yield) in dicing performed at the time of manufacturing the ultrasonic transducer was performed. In addition, a test for confirming whether or not an acoustic matching layer was peeled (durability) at the time of disinfection performed after use of an ultrasonic probe was performed.

(Preparation of Four-Layer Matching Layer)

90 parts by mass of an epoxy resin 1 (jER-630, manufactured by Mitsubishi Chemical Corporation), 5 parts by mass of an epoxy resin curing agent 1 (jER Cure WA, manufactured by Mitsubishi Chemical Corporation), 5 parts by mass of an epoxy resin curing agent 2 (CUREZOL 1B2MZ, manufactured by Shikoku Chemicals Corporation), 373 parts by mass of ferrite powder (LD-M, specific gravity 5.6, manufactured by JFE Chemical Corporation), and 322 parts by mass of tungsten powder (W-2KD, specific gravity 19.3, manufactured by JAPAN NEW METALS CO., LTD.) were sufficiently mixed using a vacuum mixer (ARV-310P, manufactured by THINKY CORPORATION) at a rotation speed of 2000 rpm and a vacuum pressure of 0.5 kPa for five minutes to prepare a compound 1.

A compound 2 was prepared in a similar manner to the compound 1 except that the types and masses of the epoxy resin, the epoxy resin curing agent, the ferrite powder, and the tungsten powder used were changed as presented in Table 3.

A compound 3 was prepared in a similar manner to the compound 2 except that 5 parts by mass of silicone rubber powder (KMP-605, specific gravity 0.98, manufactured by Shin-Etsu Chemical Co., Ltd.) was used instead of the tungsten powder, and the mass of the ferrite powder was changed to 56 parts by mass.

A compound 4 was prepared in a similar manner to the compound 3 except that the type and mass of the epoxy resin and the mass of the epoxy resin curing agent were changed as presented in Table 5, and ferrite was not added. Note that, as epoxy resins 1 to 3 and epoxy resin curing agents 1 to 3 in Table 5, the following were used.

Epoxy resin 1 (jER-630, manufactured by Mitsubishi Chemical Corporation)

Epoxy resin 2 (jER-828, manufactured by Mitsubishi Chemical Corporation)

Epoxy resin 3 (jER-807, manufactured by Mitsubishi Chemical Corporation)

Epoxy resin curing agent 1 (jER Cure WA, manufactured by Mitsubishi Chemical Corporation)

Epoxy resin curing agent 2 (CUREZOL 1B2MZ, manufactured by Shikoku Chemicals Corporation)

Epoxy resin curing agent 3 (jER Cure 113, manufactured by Mitsubishi Chemical Corporation)

TABLE 5

| | | | | | | | Coating | Film | | Sound | Acoustic |
|---|---|---|---|---|---|---|---|---|---|---|---|
| | | Epoxy | | | | | temperature | thickness | Density | speed | impedance |
| | | resin | Curing agent | | Filler | | [° C.] | [μm] | [g/cm³] | [m/s] | [MRayls] |
| Acoustic | Compound 1 | Epoxy | Epoxy resin | Epoxy resin | Ferrite | Tungsten | 80 | 60 | 4.7 | 2610 | 12.2 |
| matching | | resin 1 | curing agent 1 | curing agent 2 | powder | powder | | | | | |
| layer 1 | | 90 parts | 5 parts by | 5 parts by | 373 parts | 322 parts | | | | | |
| | | by mass | mass | mass | by mass | by mass | | | | | |
| Acoustic | Compound 2 | Epoxy | Epoxy resin | — | Ferrite | Tungsten | 40 | 50 | 3.4 | 2420 | 8.2 |
| matching | | resin 2 | curing agent 3 | | powder | powder | | | | | |
| layer 2 | | 76 parts | 24 parts by | | 280 parts | 138 parts | | | | | |
| | | by mass | mass | | by mass | by mass | | | | | |
| Acoustic | Compound 3 | Epoxy | Epoxy resin | — | Ferrite | Silicone | 30 | 50 | 1.5 | 2420 | 3.6 |
| matching | | resin 2 | curing agent 3 | | powder | rubber | | | | | |
| layer 3 | | | | | | powder | | | | | |
| | | 76 parts | 24 parts by | | 56 parts | 5 parts | | | | | |
| | | by mass | mass | | by mass | by mass | | | | | |
| Acoustic | Compound 4 | Epoxy | Epoxy resin | — | Silicone | — | 43 | 40 | 1.1 | 1770 | 1.9 |
| matching | | resin 3 | curing agent | | rubber | | | | | | |
| | | | | | powder | | | | | | |
| layer 4 | | 72 parts | 28 parts by | | 69 parts | | | | | | |
| | | by mass | mass | | by mass | | | | | | |

The compound 1 was applied onto a Teflon substrate (“Teflon” is a registered trademark of Chemours) using a blade coater (model number, manufacturer) under a temperature condition of 80° C. so as to have a thickness of 60 μm. Thereafter, the compound 1 was heated and dried in a thermostatic chamber at 100° C. for one hour to prepare an acoustic matching layer 1. The thickness of the acoustic matching layer 1 measured with an electron microscope (S-800, manufactured by Hitachi High-Tech Co., Ltd.) was 59 μm.

The compound 2 was applied onto the acoustic matching layer 1 using a blade coater under a temperature condition of 40° C. so as to have a thickness of 50 μm, thereby preparing an acoustic matching layer 2. Subsequently, the compound 3 was applied onto the compound 2 under a temperature condition of 30° C. so as to have a thickness of 50 μm, thereby preparing an acoustic matching layer 3. Furthermore, the compound 4 was applied onto the compound 3 under a temperature condition of 43° C. so as to have a thickness of 40 μm, thereby preparing an acoustic matching layer 4.

Thereafter, the laminate of the acoustic matching layers 1 to 4 was allowed to stand in a thermostatic chamber at 150° C. for three hours, sufficiently cured, and then removed from the Teflon substrate (“Teflon” is a registered trademark of Chemours) to prepare a four-layer matching layer.

(Measurement of Density of Acoustic Matching Layer)

The density of the acoustic matching layers 1 to 4 was measured using an electronic densimeter (SD-200L, manufactured by Alfa Mirage Co., Ltd.) in accordance with a density measurement method of a water substitution method described in JIS K7112 02.

(Measurement of Sound Speed of Acoustic Matching Layer)

The ultrasonic sound speed in the acoustic matching layers 1 to 4 was measured at 25° C. using a sing-around type sound speed measuring apparatus manufactured by Ultrasonic Engineering Co., Ltd. in accordance with JIS Z2353-2003.

The obtained density was multiplied by the sound speed to calculate the acoustic impedance of the acoustic matching layers 1 to 4.

(Preparation of Backing Layer)

91 parts by mass of liquid silicone rubber (TSE3032 (A), manufactured by Momentive Performance Materials Inc.) and 750 parts by mass of tungsten trioxide powder (A2-W03, manufactured by A.L.M.T. Corp.) were sufficiently mixed by a vacuum mixer (ARV-310, manufactured by THINKY CORPORATION). Thereafter, 9 parts by mass of liquid silicone rubber (TSE3032 (B), manufactured by Momentive Performance Materials Inc.) was added thereto, and the mixture was mixed with the mixer.

The obtained mixture was put into a die of 100 mm×100 mm×30 mm, allowed to stand under vacuum at room temperature (25° C.) for three hours at a pressure of 4.9 MPa with a vacuum electrothermal press machine (IMC-19AE, Imoto machinery Co., Ltd.), and then heated at 50° C. for three hours to prepare a block of composite particles. At this time, the density of the block measured by a similar method to the measurement of the density of the acoustic matching layer was 7.3 g/cm³.

The block was cut into a 1 cm square, coarsely pulverized by a cutter mill (VM-20, manufactured by Makino MFG. Co., Ltd.), and then subjected to main pulverization with a pin mill pulverizer (Model M-4, manufactured by NARA MACHINERY CO., LTD.) at a screen size of 0.5 mm and a rotation speed of 2800 rpm. Then, the resulting product was sieved with a circular vibration sieve machine (KG-400, manufactured by Nishimura Machine Works Co., Ltd.) at a mesh size of 212 μm to prepare filler composite particles. At this time, the average particle size of the particles measured with a laser type particle size distribution analyzer (LMS-30, manufactured by Seishin Enterprise Co., Ltd.) was 123 mm.

91 parts by mass of an epoxy resin (Albidur EP2240, manufactured by NANORESIN) and 380 parts by mass of the filler composite particles were sufficiently mixed by a vacuum mixer (ARV-310, manufactured by THINKY CORPORATION). To the resulting mixture, 9 parts by mass of a crosslinking agent (jER Cure ST-12, manufactured by Mitsubishi Chemical Corporation) was added, and the mixture was mixed with the vacuum mixer to prepare a resin mixture.

The resin mixture was put into a die of 100 mm×100 mm×30 mm, allowed to stand at room temperature (25° C.) for four hours at a pressure of 9.9 MPa using a vacuum electrothermal press machine, and then heated at 60° C. for three hours to form a backing block. At this time, the density of the block measured by a similar method to the measurement of the density of the acoustic matching layer was 2.65 g/cm³. In addition, an acoustic impedance obtained by multiplying the sound speed measured by the sound speed measurement of the acoustic matching layer by the density of the block was 2.9 MRayls.

Furthermore, according to JIS Z2354-1992, an ultrasonic wave attenuation ratio was calculated by filling a water tank with water at 25° C., generating an ultrasonic wave of 1 MHz in water by an ultrasonic pulser/receiver (JPR-10C, manufactured by Japan Probe Co., Ltd.), and measuring the magnitude of an amplitude before and after the ultrasonic wave passed through the resin composition. The calculated attenuation ratio was 30 dB/cmMHz. The block was cut to a thickness of 6 mm using a wire saw (CS-203, manufactured by Musashino Denshi Inc.), and then polished to a thickness of 5 mm using a precision polishing apparatus (MA-200, manufactured by Musashino Electronics Co., Ltd.) to prepare a backing layer.

(Preparation of Acoustic Lens)

Titanium oxide particles (CR60-2, manufactured by ISHIHARA SANGYO KAISHA, LTD.) were thinly placed on a stainless steel pad, and then put into a dryer at 250° C. and dried for four hours to remove surface-adsorbed water. Subsequently, 100 parts by mass of a silicon rubber compound (KE742U, manufactured by Shin-Etsu Chemical Co., Ltd.) and 40 parts by mass of the titanium oxide fine particles were kneaded using a roll kneader (No. 191-TM/WM test mixing roll, manufactured by Yasuda Seiki Seisakusho, Ltd.) to prepare a rubber composition.

Subsequently, 0.5 parts by mass of 2,5-dimethyl-2,5-di(t-butylperoxy) hexane as a vulcanizing agent was added to 100 parts by mass of the rubber composition in a roll kneader to prepare a molding compound. The obtained molding compound was press-molded at 165° C. for 10 minutes using a manual molding machine (P500F-4141, manufactured by Shoji Co., Ltd.), and then further subjected to secondary vulcanization at 200° C. for two hours to prepare an acoustic lens. Note that the acoustic lens had an acoustic impedance of 1.3 MRayls and an acoustic attenuation ratio of −0.7 dB/mm·MHz.

(Preparation of Ultrasonic Transducer)

The backing layer, an FPC, a piezoelectric material (PZT 3203HD, thickness 0.13 mm, manufactured by CTS Electro Component), and the four-layer matching layer were laminated in this order. The piezoelectric material and the four-layer matching layer were bonded to each other using the adhesive liquid A1, and the backing layer and the FPC were bonded to each other using the adhesive liquid D.

Subsequently, dicing was performed using a dicer (DAD323, thickness 0.02 mm, manufactured by DISCO Corporation) at a pitch of 0.20 mm so as not to cut an electrode of each element, and the diced laminate was further diced into three equal parts.

Thereafter, a dimer (dix-C, manufactured by kisco Co., Ltd.) was put into a parylene film forming apparatus (LABCOIER PDS2010, manufactured by SCS Corporation), and the laminate was coated with a polychloroparaxylylene film so as to have a film thickness of 3 μm. Then, an RTV silicone adhesive (KE-1604, manufactured by Shin-Etsu Chemical Co., Ltd.) was filled in a die groove formed by the above-described dicing in vacuum, and then the acoustic lens and the laminate were pressure-bonded to each other to prepare an ultrasonic transducer 1.

An ultrasonic transducer 2 was prepared in a similar manner to the ultrasonic transducer 1 except that, in bonding the piezoelectric material and the four-layer matching layer to each other, an electrode surface of the piezoelectric material was subjected to oxygen plasma treatment with a plasma cleaner (PC-1100, manufactured by Samco Corporation), a surface treatment liquid 2a was applied to the electrode surface, and then the piezoelectric material and the four-layer matching layer were bonded to each other with the adhesive liquid 0.

(Yield)

In the ultrasonic transducers 1 and 2, after the laminate was diced, whether or not the four-layer matching layer was peeled off was observed using a microscope (SZX7, Olympus Corporation). Observation results were evaluated according to the following criteria.

∘ Peeling of the four-layer matching layer was not confirmed.

× Peeling of the four-layer matching layer was confirmed.

(Durability Test)

The ultrasonic transducers 1 and 2 were immersed in ethanol for one week, and then whether or not the four-layer matching layer was peeled off was observed using a microscope. Observation results were evaluated according to the following criteria.

∘ Peeling of the four-layer matching layer was not confirmed.

× Peeling of the four-layer matching layer was confirmed.

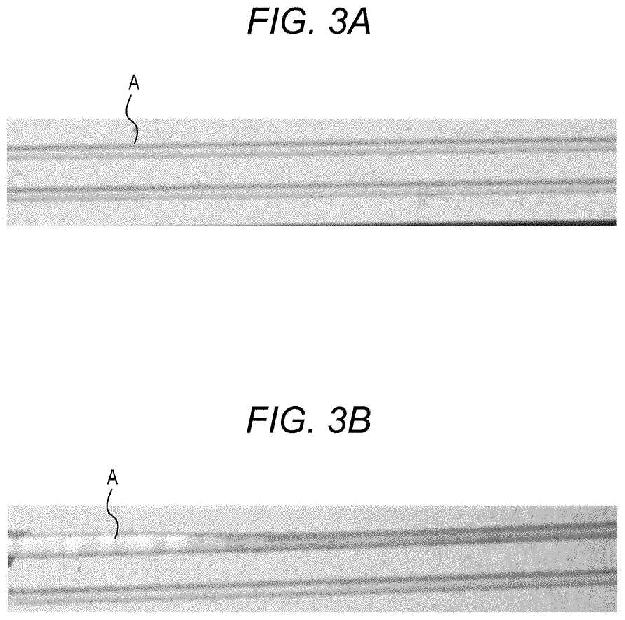

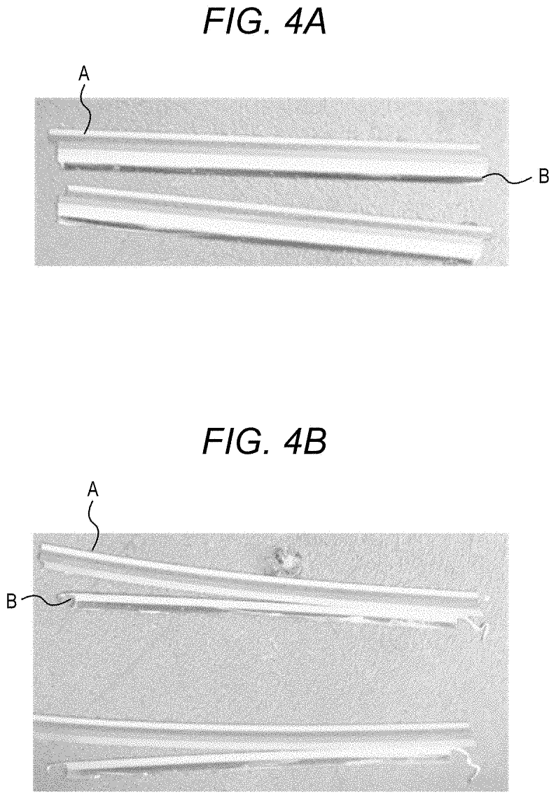

Evaluation results are presented in Table 6. FIGS. 3A and 3B illustrate images obtained by photographing states of the ultrasonic transducer 1 and the ultrasonic transducer 2 after the laminate is diced, respectively. FIGS. 4A and 4B illustrate images obtained by photographing states of the four-layer matching layers of the ultrasonic transducer 1 and the ultrasonic transducer 2 after the durability test, respectively. Note that, in FIGS. 3A, 3B, 4A, and 4B, A represents the acoustic matching layer (four-layer matching layer), and B represents the piezoelectric material. In FIGS. 3A and 3B, the piezoelectric material is disposed in a depth direction of the drawing with respect to the acoustic matching layer.

TABLE 6

Evaluation result

| | Yield | Durability |
|---|---|---|
| Ultrasonic transducer 1 | ∘ | ∘ |
| Ultrasonic transducer 2 | x | x |

The ultrasonic transducer according to the present invention can improve adhesive strength between acoustic members, and can suppress peeling of the acoustic members at the time of dicing and peeling of the acoustic members at the time of disinfection. Therefore, the present invention is useful, for example, in the field of ultrasonic diagnosis.

Although embodiments of the present invention have been described and illustrated in detail, the disclosed embodiments are made for purposes of illustration and example only and not limitation The scope of the present invention should be interpreted by terms of the appended claims.

What is claimed is:

1. An ultrasonic transducer comprising:
a laminate in which a plurality of acoustic members is laminated; and
an adhesive layer that comprises a silane coupling agent and an adhesive, the adhesive layer joining any two of the plurality of acoustic members to each other, wherein
the silane coupling agent has a structure represented by general formula (1): 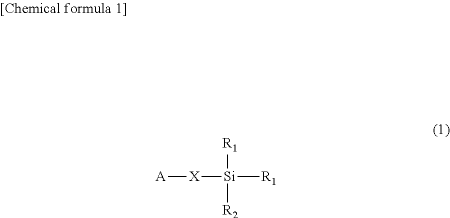
wherein R₁s each independently represent a methoxy group or an ethoxy group, R₂ represents a methoxy group, an ethoxy group, or a hydrogen atom, X represents a linear or branched organic chain having 4 or more continuous carbon atoms, and A represents a reactive functional group.

2. The ultrasonic transducer according to claim 1, wherein the adhesive layer comprises an organic acid having 2 or more and 6 or less carbon atoms or a salt thereof.

3. The ultrasonic transducer according to claim 1, wherein in the general formula (1), A is at least one functional group selected from the group consisting of a mercapto group, a vinyl group, an acryloyl group, an epoxy group, an amino group, and an isocyanate group.

4. The ultrasonic transducer according to claim 1, wherein
the acoustic member comprises an acoustic matching layer, and
the adhesive layer joins the acoustic member to another acoustic member.

5. The ultrasonic transducer according to claim 4, wherein
the acoustic member comprises a piezoelectric material that transmits and receives an ultrasonic wave, and
the adhesive layer joins the piezoelectric material to the acoustic matching layer.

6. The ultrasonic transducer according to claim 4, wherein the acoustic matching layer comprises elastomer particles.

7. The ultrasonic transducer according to claim 4, wherein the acoustic matching layer is formed by laminating a plurality of acoustic matching layers.

8. The ultrasonic transducer according to claim 7, wherein the plurality of acoustic matching layers is formed by laminating four or more acoustic matching layers.

9. The ultrasonic transducer according to claim 7, wherein among the plurality of acoustic matching layers, a layer farthest from a piezoelectric material in an ultrasonic wave propagation direction comprises elastomer particles.

10. The ultrasonic transducer according to claim 4, wherein the acoustic matching layer comprises a layer comprising a resin and particles having a specific gravity of 4.5 or more and 6.0 or less, and a content of the particles is 150% by mass or more and 1200% by mass or less with respect to a total mass of the resin in the layer comprising the resin and the particles.

11. The ultrasonic transducer according to claim 1, wherein the adhesive comprises an epoxy resin.

12. The ultrasonic transducer according to claim 1, wherein the adhesive has a glass transition temperature of 60° C. or higher.

13. The ultrasonic transducer according to claim 1, wherein the adhesive layer has a thickness of 1 μm or less in an ultrasonic wave propagation direction.

14. An ultrasonic probe comprising the ultrasonic transducer according to claim 1.

15. An ultrasonic diagnostic apparatus comprising the ultrasonic probe according to claim 14.

16. A method for manufacturing an ultrasonic transducer, the method comprising:
disposing an adhesive layer comprising a silane coupling agent and an adhesive on a surface of at least one of a plurality of acoustic members; and
laminating another acoustic member on the surface on which the adhesive layer is disposed, wherein
the silane coupling agent has a structure represented by general formula (1): 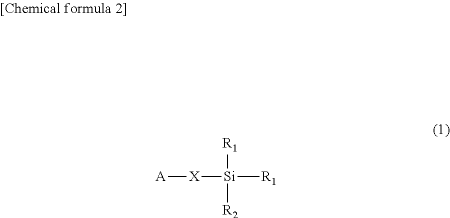
wherein R₁s each independently represent a methoxy group or an ethoxy group, R₂ represents a methoxy group, an ethoxy group, or a hydrogen atom, X represents a linear or branched organic chain having 4 or more continuous carbon atoms, and A represents a reactive functional group.

17. The method for manufacturing an ultrasonic transducer according to claim 16, wherein, in the disposing of the adhesive layer, the adhesive comprising the silane coupling agent is disposed on the surface of the acoustic member.

18. The method for manufacturing an ultrasonic transducer according to claim 16, further comprising subjecting a surface of the acoustic member to oxygen plasma treatment.
